(19) United States
(12) Patent Application Publication
(10) Pub. No.: US 2007/0122398 A1
(43) Pub. Date: May 31, 2007

(54) Encapsulated hydrophilic compounds

(76) Inventors: Daniel Benczedi, Plan-Les-Ouates (CH); Alexander Hahn, Singapore (SG); Gil Trophardy, Gex (FR); Ennio Cantergiani, Blonay (CH); Robert Wagner, Sergy (CH)

Correspondence Address: WINSTON & STRAWN LLP;PATENT DEPARTMENT, 1700 K STREET, N.W., WASHINGTON, DC, 20006, US

(21) Appl. No.: 11/643,286

(22) Filed: Dec. 20, 2006

Related U.S. Application Data
(63) Continuation of application No. PCT/IB05/01779, filed on Jun. 23, 2005.

(30) Foreign Application Priority Data
Jul. 2, 2004 (EP) 04103143.6

(51) Int. Cl. A61K 35/74; A61K 47/00; A61K 9/48

(52) U.S. Cl. 424/93.45; 424/439; 424/451

(57) ABSTRACT

The present invention relates to capsules for encapsulating functional agents, such as flavors, fragrances, pharmaceuticals, vitamins, etc. The capsules are suitable for the encapsulation of hydrophobic as well as hydrophilic substances. The capsules include a micro-organism, a matrix component and the encapsulatable material, wherein the latter comprises the functional agent or agents. The invention further relates to a process for manufacturing the capsules and to food products containing the capsules.

### CROSS-REFERENCE TO RELATED APPLICATION

This application is a continuation of International application PCT/IB2005/001779 filed Jun. 23, 2005, the entire content of which is expressly incorporated herein by reference thereto.

### TECHNICAL FIELD AND PRIOR ART

The present invention relates to capsules comprising micro-organisms, a delivery system or a food product comprising the capsules and to a method for manufacturing the capsules.

The delivery of functional agents, ingredients, molecules or compositions such as flavors, fragrances, pharmaceuticals, herbicides and many others is an issue with nearly all applied sciences. Without the stabilization of a concentrated, easily transportable and processable form of the functional agent delivery becomes unreliable and the functional agents will only rarely exhibit their beneficial properties at the predetermined place and time.

Encapsulation is key when it comes to the delivery of stabilized functional agents, and many different encapsulation technologies and systems have been developed so far. The encapsulation of micro-organisms was disclosed in U.S. Pat. No. 4,001,480 and offered a number of advantages, such as the utilization of a inexpensive raw material, the micro-organism, for providing a solid capsule for lipophilic substances, enclosed within the cell walls of the micro-organism. An important advantage of the resulting microbe-based capsules is the controlled release. The dye was retained in the capsule until its liberation was effected. Accordingly to the method, yeast was grown in a specific medium in order to obtain yeasts with high lipid content. The functional agent, a dye, was then dissolved in a carrier, ethyl alcohol, and brought in contact with the yeast biomass. After incubation for a few minutes the yeast cells were observed as being infused with the dye. The delivery system so created was useful as a coloring agent. This process had the disadvantages that only fungi having a natural fat content of 40 to 60% could be used, which required very specific growing procedures.

In EP 0 085 850 the encapsulation in microbes having less than 40 wt. % of lipid content was postulated, however, a lipid extending substance had to be employed, defined as a substance which is miscible with the microbial lipid and which is capable of diffusion through the cell wall of the microbe. The functional agent to be encapsulated, again a dye, was dissolved in the lipid-extending substance. This solution was mixed into an aqueous slurry of yeast cells and stirred until diffusion of the solution, including the dye, into the yeast cells.

The constraint of using a lipid-extending substance could be removed following the teaching of EP 0 242 135 A2, where certain lipophilic substances, such as cedar oil, mint oil, peppermint oil, eucalyptus oil, malathione, and others were shown to diffuse across the microbial cell wall and to be retained passively within the microbe.

The mechanisms and kinetics of the accumulation of essential oils by yeast cells were further studied by Bishop et al, “Microencapsulation in yeast cells”, J. Microencapsulation, 1998, 15, No. 6, 761-773, who found that the rate of permeation of oil into the yeast cells increased significantly at higher temperatures due to the phase transition of the lipid membrane of the cells. The cells lost quickly viability during the process and it appeared unnecessary for the cells to be viable for the process to occur.

It was found that the process of the prior art suffers from the drawback that during the drying and/or centrifugation process of the encapsulated yeast, a significant amount of functional agent, flavors, etc, is lost, especially the volatile ones. There is thus a need to provide a capsule wherein even volatile functional agents can subsist for prolonged time.

WO 03-041509 discloses microcapsules having a foreign material enclosed in microbial cells, wherein at least one member of the group consisting of saccharides, sweeteners, proteins and polyhydric alcohols is adhered to the surface of the microorganisms.

The significant drawback of the methods of encapsulation of the prior art is that they are not suitable to encapsulate functional agents that are more hydrophilic than oils, for example, because hydrophilic agents are not retained in the plasma of the yeast cell after having freely defused through the cell wall.

In other words, there is a need for an encapsulation system for hydrophilic functional agents.

In addition, there is a need for an encapsulation system containing both, hydrophobic and hydrophilic functional agents. For example, one can envisage a delivery system containing two pharmaceuticals, one of which being hydrophilic and one of them hydrophobic, which are designed for concomitant application to a patient. In this example, the micro-capsules according to the prior art disclosed above would not be suitable, because only the hydrophobic one could diffuse into yeast cells in the above described methods.

Flavoring or fragrance ingredients, in particular, are often composed of a multitude of different individual compounds, which altogether are responsible for a specific aroma or fragrance profile or for a specific taste. The different flavor compounds that make up a specific flavor composition may have different chemical structures and solubility parameters, which explains why the yeast encapsulation systems of the prior art are not useful, largely discriminating hydrophilic flavor or fragrance compounds. In the case of flavoring compositions, this results in micro-capsules which may provide a different, sometimes even less preferred, for example unbalanced, taste due to the absence of hydrophilic flavor components.

Therefore, there is a need for a delivery system suitable to provide an original flavor or perfume profile, preserving the roundness of a selected composition of different flavor or perfume compounds.

In addition, there is a need for controlling the release of the functional agents contained within capsules. The functional agent, if it is volatile, for example, should be retained as long as necessary within the capsule.

In short, there is a need for capsules that allow a controlled release of the functional agent or the mixture of functional agents contained therein.

### SUMMARY OF THE INVENTION

Remarkably, the inventors have found a surprising way of encapsulating also hydrophilic flavor compounds into capsules based on micro-organisms. The present invention thus enables the encapsulation of functional agents of different hydrophobicity in a single capsule.

Accordingly, the present invention provides, in a first aspect, capsules comprising a micro-organism, a matrix component, and, at least one encapsulatable material, whereby the matrix component and the encapsulatable material do not originate from the micro-organism itself, and whereby the encapsulatable material comprises at least one functional agent that is characterized by a calculated octanol/water partition coefficient clogP smaller than 3.

In a second aspect, the present invention provides a delivery system comprising the capsules of the present invention.

In a third aspect, the present invention provides a food product comprising the capsules of the present invention.

In a fourth aspect, the present invention provides a process for preparing the capsules according to the present invention, comprising the steps of
preparing an aqueous liquid comprising at least a micro-organism and water,
adding an encapsulatable material comprising a functional agent having a clogP of smaller than 3,
stirring, agitating or mixing the aqueous liquid and the encapsulatable material,
adding a matrix component
drying the components, and, optionally,
granulating the dried slurry to obtain the capsules according to the present invention.

### BRIEF DESCRIPTION OF THE DRAWING FIGURES

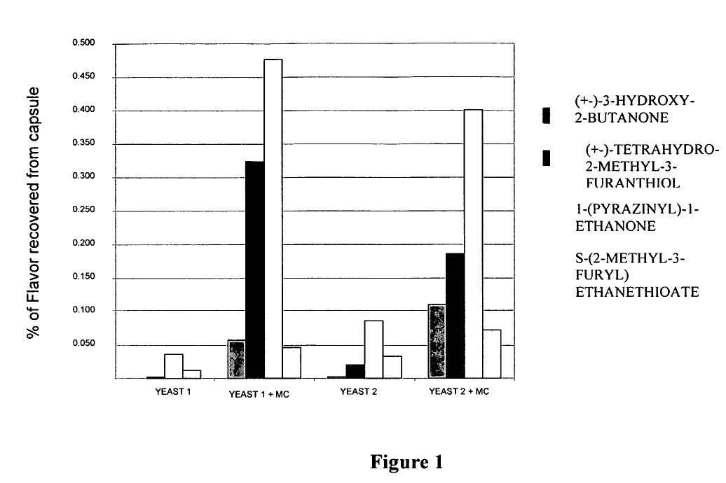

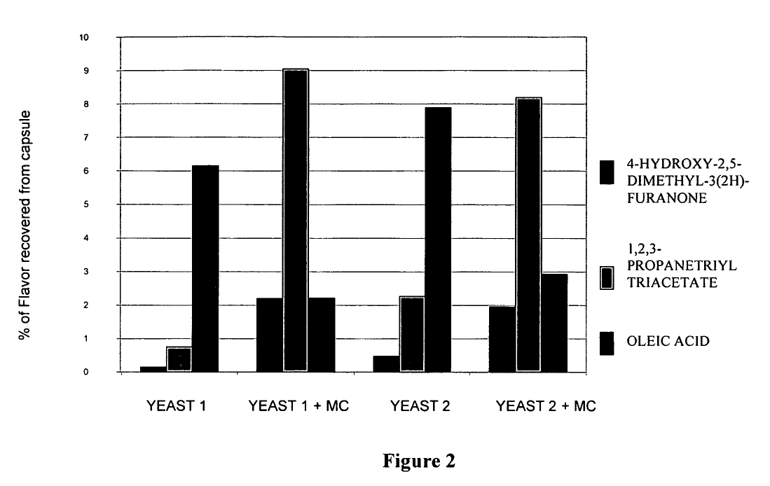

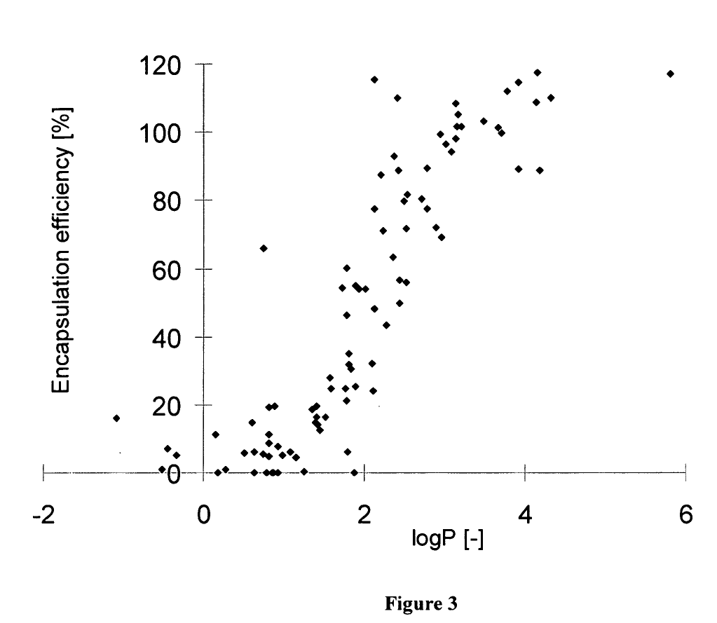

In the figures, FIG. 1 and 2 show the percentage of recovered flavor from different capsules, with respect to the flavor used in the process of preparation. The traditional yeast-encapsulation is thereby compared to the encapsulation including a matrix component according to the present invention. A range of different flavors having different clogP values were encapsulated. FIG. 3 shows encapsulation efficiency of yeast—flavor microcapsules in the absence of a matrix component as a function of clogP values. It can be seen that in the absence of a matrix component, flavor compounds with a clogP of <3 or even <2 become increasingly difficult to encapsulate by the yeast-based system alone.

### DETAILED DESCRIPTION OF THE PREFERRED EMBODIMENTS

In the context of the present invention, percentages are percentages by weight of dry matter, unless otherwise indicated. Similarly, if proportions are indicated as parts, parts of weight of dry matter are meant.

The term “mean” as used, for example in the expression “mean diameter” refers to the arithmetic mean.

The term logP refers to the octanol/water partition coefficient of a specific functional agent to be encapsulated. For the purpose of the present invention, reference to a calculated logP (often abbreviated as clogp) value is made. This value is calculated by the software T. Suzuki, 1992, CHEMICALC 2, QCPE Program No 608, Department of chemistry, Indiana University. See also T. J. Suzuki, Y. Kudo, J. Comput.-Aided Mol. Design (1990), 4, 155-198. The clogP value is widely used by the industry, because it allows to reliably attribute a logP value to any compound in a short time.

The term, “functional agent” is not restricted to a specific class of molecules. It refers to a substance, a compound, and/or an ingredient, for example. The functional agent is defined as the part of the capsule that is intended to be delivered due to its function, while other parts of the capsule are usually used as carriers or ingredients for stabilizing the functional agent or controlling its release. A list of suitable functions is given further below (flavors, etc.). In practice, the function or purpose of the functional agent is often indicated on the packaging containing the capsules of the present invention. The function can be performed by one or more functional agents. Similarly, several functions may be performed by different functional agents contained in the same capsule.

The present invention provides capsules comprising a matrix component and encapsulatable material both of which do not originate from the micro-organism, which is also part of the capsules. The term “do not originate” from is used to clarify that the matrix component and the encapsulatable material are parts of the capsules, which were added, during the process of manufacture, as individual components. They are not part of the micro-organism foreseen for encapsulation as it is found in its native state. For the avoidance of doubt, however, it is stated that the matrix component and/or the encapsulatable material may, theoretically, be isolated from micro-organisms and then be added to the micro-organisms of the present invention. This is true, for example, for some polysaccharides, which may be harvested from micro-organisms and which may then be used as matrix component in the capsules of the invention. Similarly, many flavors are obtained in fermentation processes and are thus the product of a micro-organism, which can be used as encapsulatable material for encapsulation in the capsule of the present invention as an individual component.

The present invention provides capsules comprising a micro-organism, and amongst other components, encapsulatable material that comprises a functional agent having a calculated octanol/water coefficient (clogp) of smaller than 3.

In a preferred embodiment, the functional agent is characterized by a clogP of smaller than 2. Preferably, the clogP is smaller than 1.5, more preferably smaller than 1, most preferably, smaller than 0.5.

Preferably, the lower limit of the clogP value for the functional agent of the present invention is −3, more preferably −2.5, most preferably −2. For example, the functional agent of the present invention may have a clogP in the range of −3 to 3.

The functional agent can be selected from all sorts of functional agents. They can be food additives, such as taste enhancers, aromas, flavors, for example. Other functional agents are fragrances, pharmaceuticals, vitamins, herbicides, fungicides, insecticides, detergents, cleaning agents, liquid bleach activators, dyes, just to mention a few functions.

In a preferred embodiment of the present invention, the functional agent with clogP <3 is a flavor, an aroma or a fragrance. More preferably it is a flavor.

The term “flavor” is meant a compound, which is used alone or in combination with other compounds, to impart a desired gustative effect. To be considered as a flavor, it must be recognized by a skilled person in the art as being able to modify in a desired way the taste of a composition. Such compositions are intended for oral consumption and are hence often foods, nutritional compositions and the like.

The textbook “Perfume and Flavor Chemicals” Steffen Arctander, published by the author, 1969, is a collection of perfumes and flavors known to the skilled person and is expressly incorporated herein in its entirety by reference. The molecules of this textbook are suitable for being encapsulated in the capsules of the present invention, provided that they fulfill the clogP-requirements of the invention.

The functional agent may be a mixture of different flavors. This has the advantage that the capsules of the present invention provide a rounded, composed flavor, giving a more versatile, complete flavor and/or fragrance impression upon consumption.

The possible flavors to be provided by the functional agents are the flavors associated with meat, such as beef, chicken, pork, or with fish, for example. The flavor may be associated with vegetables, fruits, berries, for example. The flavor may be a spice or a composition of spices.

Table 1 contains an exemplary list of functional agents suitable for the present invention. The functional agent is identified by its systematic name as well as its clogP value. The function of each agent is also indicated in most cases.

TABLE 1
Functional agents suitable for encapsulation in the capsules of the present invention

| Functional agent | clogP | Flavor function |
|---|---|---|
| (+−)-3-HYDROXY-2-BUTANONE | −0.5 | dairy note, sour cream, butter |
| (+−)-TETRAHYDRO-2-METHYL-3-FURANTHIOL | 0.83 | meaty |
| 1-(PYRAZINYL)-1-ETHANONE | −0.33 | roasted, bred crust |
| S-(2-METHYL-3-FURYL) ETHANETHIOATE | 2.11 | meaty |
| 4-HYDROXY-2,5-DIMETHYL-3(2H)-FURANONE | 0.28 | cotton candy |
| 1,2,3-PROPANETRIYL TRIACETATE | −1.36 | |
| 5-ALLYL-4,7-DIMETHOXY-1,3-BENZODIOXOLE | 1.65 | |
| 6-ALLYL-4-METHOXY-1,3-BENZODIOXOLE | 2.18 | spicy, nutmeg |
| (Z)-3-HEXEN-1-OL | 1.49 | freshly cut grass |
| 1,8-cineole (1,8-EPOXY-P-MENTHANE) | 2.63 | camphorous |
| Camphor | 2.22 | camphorous |
| (1,7,7-TRIMETHYL-BICYCLO[2.2.1]HEPTAN-2- | | |
| ONE) | | |

Preferably, the functional agent is selected from the group consisting of the flavors listed in Table 1.

The capsule according to the present invention further comprises a matrix component. The matrix component is preferably suitable to form a polymer matrix. There are a vast number of structurally different matrix-forming compounds or compositions, some of which are mentioned below.

The matrix component may, for example, be formed of or comprise a protein. Suitable matrix components are caseins, whey proteins, and/or soy protein. Preferably, the matrix component may be gelatin. These proteins have good emulsification and film forming properties and can form the basis for polymer matrices providing elevated retention and protection of volatile functional agents.

The matrix component may comprise carbohydrates. In an embodiment of the present invention, the carbohydrate is water soluble. The term “soluble fiber” means that the fiber is at least 50% soluble according to the method described by L. Prosky et al., J. Assoc. Off. Anal. Chem. 71, 1017-1023 (1988).

The matrix component may, besides a water-soluble carbohydrate, additionally contain a carbohydrate, which is not soluble in water, in order to modify the matrix properties as desired. For example, the matrix component may further contain cellulose and/or hemi-cellulose, in addition to a soluble carbohydrate.

For example the matrix component may comprise monosaccharides, for example, D-Apiose, L-Arabinose, 2-Deoxy-D-ribose, D-Lyxose, 2-O-Methyl-D-xylose, D-Ribose, D-Xylose, which are all Pentoses or Hexoses like for instance L-Fucose , L-galactose, D-Galactose, D-Glucose, D-Mannose, L-Rhamnose, L-mannose, or mixtures of several of these.

Similarly, dissacharides, trisaccharides and tetrasaccharides are possible useful matrix components.

Mono- and dissacharides may be reduced to the corresponding alcohols like for example xylitol, sorbitol, D-mannitol and/or maltitol, for example. Similarly, oxidation to aldonic, dicaroxyclic acids or uronic acids and reactions with acids, alkalis or amino compounds can give rise to many other compounds like isomaltol, for instance, which may be comprised in the matrix component of the present invention.

The matrix component may comprise mixtures of the above- and/or below mentioned carbohydrates, their derivatives and/or proteins. For example, mono-di or trisaccharides and/or their reaction products (see above) may be used as additives in combination with a protein or polysaccharide based matrix and thus bring properties as desired to the matrix component.

The matrix component may comprise oligosaccharides, that is, molecules consisting of from 3-10 monosaccharide units. Examples are maltopentaose, fructo- and/or galactooligosaccharides.

Preferably, the matrix component comprises polysaccharides, that is, saccharides containing more than 10 monosaccharide units per molecule.

These polymers can be either perfectly linear (cellulose, amylose), branched (amylopectin, glycogen) or linearly branched. They can include carboxyl groups (pectin, alginate, carboxymethyl cellulose) or strongly acidic groups (furcellaran, carrageenan or modified starch). They can be modified chemically by derivatization with neutral substituents (in the case of methyl ethyl cellulose or hydroxypropyl cellulose for instance) or acidic substituents (with carboxymethyl, sulfate or phosphate groups).

More preferably, the matrix component comprises a starch derivative. This group of polysaccharides itself includes a lot of different polymers since it is possible to modify the starch either by mechanically damaging the starch granules (grinding or extrusion), by heating with or without an acid or a base to pre-gelatinized it or degrade it to get thin- or thick- boiling starch, dextrins or maltodextrins of various molecular weights. Other possible modifications of starch and resulting derivatives include octenyl-succinated starch, starch ethers (i.e. carboxymethyl starch), starch esters (i.e., starch monophosphate), crosslinked starch and/or oxidized starch.

Preferably, the matrix component comprises dextrin, more preferably maltodextrin and/or corn syrup. Most preferably, the matrix component comprises maltodextrin and/or corn starch syrup having a mean dextrose equivalence of 5-25, preferably 6-20, more preferably 10-18.

Likewise, the matrix component may comprise gums and/or hydrocolloids, for example, like gum arabic, gum tragacanth, karaya gum, seaweed or shell extracts like agar, carrageenan, fucoidan, alginic acid, laminaran, furcellaran and/or chitosan, or microbial polysaccharides like dextran, pullulan, elsinan, curdlan, scleroglucan, levan, xanthan, gellan, welan gum and rhamsan gum.

In addition, gum ghatti, gum, karaya gum, laminaran or pectins may be used in the formulation of the matrix component.

The matrix component may or may not comprise flurther yeast derived material, which does not contain encapsulatable material, such as, for example, yeast derived carbohydrates, but which may be used for adding further dry matter to the aqueous liquid and the encapsulatable material once encapsulation has been completed and prior to drying. Preferably, the matrix component comprises less than 90%, more preferably less than 70%, still more preferably less than 50% and most preferably less than 25% by weight of further yeast material in the matrix component. Preferably, the matrix component is free of yeast material added after encapsulation.

The exemplary list of matrix components given above illustrates the wide applicability of the present invention. The matrix component may consist of only one, particularly suitable, component, or from a mixture of two or more of such components, possibly admixed with further ingredients, for example for modifying the parameters such as permeability, mechanical strength and/or solubility, of the matrix component as desired.

The capsules according to the present invention comprise a micro-organism. The purpose of the micro-organism is the encapsulation of the optionally present, more hydrophobic functional agents, having a clogP value of 1.5, 2, 3, 4 or higher.

In a preferred embodiment of the present invention, the micro-organism is selected from the group consisting of fungi, a bacteria, algae, protozoa, or mixtures of two or more of these. Candidates of micro-organisms suitable for the purpose of the present invention are found in the prior art for example, EP 0 085 805 B1, col. 2, lines 15-25; or, EP 0 242 135A2, page 2, lines 37-40; or, EP 0 453 316 Al, col. 5, lines 20-30. The cited text positions are expressly incorporated herein by reference. Preferably, the micro-organism is a fungus or a bacterium, more preferably it is a yeast. Suitable yeast is commercially obtainable.

The micro-organism may be pre-treated for increasing its permeability for the encapsulatable material, for example, or for removing the sometimes undesired odor or aroma of the micro-organism, for example. Such pre-treatments are disclosed in U.S. Pat. No. 5,521,089, col. 2, line 58 to col. 4, line 63 and WO 93/11869. In this latter reference, a peroxygen bleaching of micro-organisms for removing odor and lightening the color of micro-organisms is disclosed.

Accordingly, in a preferred embodiment of the present invention, the capsules comprise at least one additional functional agent, which is characterized by an octanol/water partition coefficient clogP of 1 or higher, preferably 1.5 or higher. In a further embodiment of the present invention the additional functional agent has a clogP of 2 or higher, more preferably 2.5 or higher, most preferably the clogP of the additional functional agent is ≧3.

Preferably, within the capsule of the invention, the additional functional agent is encapsulated within the micro-organism.

Examples for additional functional agents can be selected amongst flavors, fragrances, pharmaceuticals, etc, as indicated above for the mandatory functional agent having generally a lower clogP value.

In a preferred embodiment of the present invention, the optional, additional other functional agent with clogP ≧1 is a flavor, an aroma or a fragrance. Preferably, it is a flavor. Examples of flavors suitable for being encapsulated may selected from the group consisting of oleic acid (clogP =7.74), caryophyllene ((−)-(IR,9S,E)-4,11,1 1-TRIMETHYL-8-METHYLENE-BICYCLO [7.2.0]UNDEC-4-ENE, clogP =6.39), alpha-pinene (2,6,6-TRIMETHYL-BICYCLO[3.1.1]HEPT-2-ENE, clogP =4.32), paracymene (1-ISOPROPYL-4-METHYLBENZENE, clogP =4.19), linalol (3,7-DIMETHYL-1, 6-OCTADIEN-3-OL, log P =3.06), estragol (l-ALLYL4-METHOXYBENZENE, clogP =3.00), thymol (2-ISOPROPYL-5-METHYLPHENOL, clogP =3.38), caravacrol (5-ISOPROPYL-2-METHYLPHENOL, clogP =3.38), for example. Suitable additional functional agents may also be selected from the flavors and fragrances of the textbook of Arctander, 1969, mentioned above, provided that they fulfill the clogP requirement given above.

Preferably, the clogP of the additional, other functional agent does not exceed 8, more preferably it does not exceed 7.5, most preferably it does not exceed 7.

In fact, if two functional agents are present in the capsule of the present invention, one of them may have a relatively low and the other a relatively high clogP value. There is an area of overlap, however, in the range of clogP of 1-3, in which the functional agents are partially but not totally retained within the micro-organism, the other part being retained in the matrix component. As a consequence, the present invention also envisages that the functional agent and/or the further functional agent have one alone or both a clogP value in the range of 1 to 3, preferably 1.5 -2.5, for example 1-2.

The capsule of the present invention may, of course, comprise a multitude of different functional agents, such as flavors, for example, having all different clogP values. The present invention differs from the prior art in that a matrix component is present, in which the more hydrophilic agents are principally retained, while the more hydrophobic agents, for example the additional functional agent, are principally retained within the micro-organism. The present invention thus provides capsules, which can efficiently deliver hydrophobic and hydrophilic functional agents, and even agents, which are in the middle range of clogP 1-3. Thus, almost the whole spectrum of possible clogP values may be covered by the at least one functional agent and the at least one optional, additional functional agent. Compositions of 1-100, preferably 2-50 different functional agents may be present in the capsules of the present invention. In case of flavors, very complex and balanced flavor compositions may thus be encapsulated within the same capsules.

Preferably, the capsules of the present invention comprise at least two functional agents, one of them having a clogP value smaller than 3 and the other one having a clogP value of 3 or higher.

In a preferred embodiment of the capsules of the present invention, the encapsulatable material further comprises a carrier. Preferably the carrier is liquid at a temperature of 20° C. Preferably the carrier is a solvent for the fumctional agent. The carrier is used for the functional agent, in particular to dissolve it, transport it into the micro-organism and/or matrix component and/or dilute it. Depending on the exact solubility of the at least one functional agent, a suitable carrier for the agent may be selected. In the literature, examples of carriers are discussed. In this context, EP 0 242 135 A2, page 3, line 50 to page 3, line 4 is expressly incorporated herein by reference. Similarly, the so-called lipid-extending substances mentioned in EP 0 085 805 B1, starting from col. 2, line 27 extending to col. 4, line 25 may serve as carriers. In EP 0453 316 A1, hydrophobic liquids to be encapsulated are discussed in the paragraph of col. 5, lines 39-53. It is well explained in the following, col. 5, line 54 to col. 6, line 5 of the same reference, that the hydrophohobic liquids may be used to dissolve dyes, perfumes etc. All the above text positions are expressly incorporated herein by reference. The carrier, if present, is preferably selected from the group of alcohols, glycols, esters, aromatic hydrocarbons, aromatic lipophilic oils, carboxylic acids, alcohols, oils, fats and/or mixtures of these components. Preferably, the carrier is a lipid. More preferably, it is a fat and/or an oil. Preferably, the carrier has the food grade status and fulfils the GRAS requirement (generally regarded as safe). Of course, the carrier has to be selected to be miscible with or emulsifiable within the at least one functional agent.

In practice, many natural isolates or extracts comprising one or more functional agents, such as flavors, within a carrier, as a direct consequence of the isolation or purification procedure. For example, some extraction procedures directly yield oils containing different flavor and/or fragrance compounds, which may then directly be used as encapsulatable material according to the present invention. An example is citrus oil, which upon extraction from the rind and/or the pith of the citrus fruit by cold expression can directly be used as encapsulatable material according to the present invention.

In a preferred embodiment of the capsules of the present invention the micro-organism provides 5 to 80%, the matrix component provides 5 to 80% and the encapsulatable material comprising at least one functional agent provides 5 to 60% of the dry weight of the capsule.

Preferably, the micro-organism provides 15 to 40%, the matrix component provides 15 to 40% and the encapsulatable material comprising at least one functional agent provides 10 to 50% of the dry weight of the capsule.

Most preferably, the capsule may comprise 20 wt.-% of micro-organism, 40wt.-% of encapsulatable material and 20 wt.-% of matrix component.

For example, the encapsulatable material comprises at least one functional agent with clogP <3 the functional agent providing 10 to 40 wt.-% of the capsule and at least one additional, different functional agent providing 10 to 40 wt.-% of the capsule.

In a preferred embodiment the capsules according to the present invention have a mean diameter in the range of 5 μm to 2 mm. Preferably, the diameter is in the range of 40 μm to 1 mm, more preferably 60 μm to 500 μm.

The present invention provides a delivery system comprising the capsules of the present invention. The delivery system may consist of the capsules as such, which preferably form a powder. Such a powder can easily be incorporated into any desired product, such as a food product, a pharmaceutical product, a body care product, for example.

The delivery system of the present invention may, on the other hand, comprise other components, such as other capsules providing other functions, or simply carrier substances suitable to alleviate the storage and/or processing of the capsules of the invention and/or its application to consumer end products.

The present invention provides a food product comprising the capsules. Such a food product may be a chilled or a frozen product. It may be a food product for consumption at chilled, ambient and/or at elevated temperatures.

Preferably, the food product is an edible product as disclosed in the European patent application with the application number EP04100069.6, filed on Jan. 12, 2004 in the name of Firmenich SA. The microcapsules disclosed in this reference may simply be replaced in a ratio of 1:1 by the capsules of the present invention. Accordingly, the edible products of EP04100069.6 comprise the capsules of the present invention, and are subjected to a thermal treatment of at least 70, preferably 100, more preferably at least 170° C.

Any food processing technology suitable to apply the thermal treatment (hot temperature) to the edible product may be used, some of which are disclosed on page 6, line 17-32 of EP04100069.6 as filed. This text position is incorporated herein by reference.

By way of examples, the edible products into or onto which the capsules of the present invention can be applied include applications in high water activity such as soups; baked products such as crackers, bread, cakes; high boiled applications such as fresh and dry pasta; cereal flakes, extruded snacks, fried products such as French fries or fabricated potato chips. Preferably, the food product of the present invention refers to potato chips and/or French fries.

Depending on the nature of the food product comprising the capsules of the present invention, the technology of applying the capsules to the product may be selected. For example, if the food product is a dough-based product, the capsules may simply be mixed together with the further ingredients of the dough before the thermal treatment, such as baking. In other application, it may be useful to mix the capsules of the present invention with water and preparing a batter before applying them to a food product before thermally treating it. If the food product is French fries, for example, the capsules of the invention may be mixed with water to obtain a batter, for example, in a Hobart mixer, and coated onto French fries before par-frying at about 180° C. for 60 s in palm oil, such as disclosed on page 9, lines 17-22 of EP04100069.6 as filed.

The present invention provides a process for preparing the capsules. Accordingly, in one step, an aqueous liquid comprising at least a micro-organism and water is prepared in a suitable vessel, for example a mixer. For example dried yeast, which is commercially available, may be mixed with water. Preferably, the aqueous liquid comprising the micro-organism and water is a suspension of 10-30, preferably 15-25 wt.-% solids, depending on type of organism and equipment used.

An aqueous liquid in the context of the present invention encompasses mixtures of water and micro-organisms, and, after a further process step also the encapsulatable material. These mixtures may be suspensions, slurries, emulsions, dispersion and the like. The term “aqueous liquid” thus only specifies that water is present.

In a step of the process, the encapsulatable material comprising at least one functional agent having a clogP of smaller than 3 is added. Of course, the encapsulatable material could also be added to the water before adding the micro-organism. The addition of the encapsulatable material may entail the formation of an emulsion, depending on the hydrophobicity of the encapsulatable material. Accordingly, emulsifiers, surfactants and/or stabilizers may also be added to the aqueous liquid, for example.

In an embodiment, the process of the present invention comprises the further step of adding an encapsulatable material to the aqueous liquid comprising a micro-organism and water, whereby the encapsulatable material comprises an additional, other functional agent having a clogP of 1 or higher.

If the capsules are intends to comprise an additional, other functional agent having a clogP value of 1, 2, 3 or higher, this functional agent is preferably comprised also in the encapsulatable material comprising the functional agent having a lower clogP value. Preferably, the encapsulatable material, which is added according to the step given above, comprises all functional agents of various clogP values.

Preferably, the dry-weight ratio of micro-organism to encapsulatable material in the aqueous liquid is in the range of 1:1 to 5:1, preferably 1.4:1 to 4:1, more preferably 1.6:1 to 3:1, most preferably 1.9:1 to 2.9:1. For example the ratio is 2.1:1.

The aqueous liquid comprising the micro-organism, water and the encapsulatable material is then mixed, stirred or agitated for 1 to 6, preferably 1.5 to 5, more preferably 2 to 4 hours. This preferably happens at above-ambient temperatures, such as at above 25, preferably above 35° C., more preferably above 40° C.

During the mixing step, at least part of the encapsulatable material may defuse into the cell of the micro-organism. If the clogP of the functional agent is above about 3, a significant proportion of the functional agent will pass freely into the cells. If the clogP of a functional agent present in the encapsulatable material is lower than about 3, only a smaller portion will pass into the cells. The remaining portion will remain in the aqueous liquid outside the cells.

The general principle of the above-depicted process of encapsulation of hydrophobic compounds into a micro-organism is disclosed in EP A2 0 242 135, or in other prior art references cited earlier. However, the prior art is completely silent on the relationship between hydrophilicity and diffusion of the encapsulatable material into the cells.

Following the more or less complete diffusion of the encapsulatable material into the cells of the micro-organism (depending on the clogp), the matrix component is added.

Preferably, 0.4 to 4 parts of matrix-component are added per part of micro-organism added earlier. More preferably, 0.6 to 2, most preferably 1 part of matrix component is added for every part of micro-organism.

The weight proportions of micro-organism : encapsulatable material : matrix component of the capsules of the present invention preferably are 1:1 -5:0.4 -4, preferably 1:1.4 -4:0.6 -2.

After adding the matrix component to the aqueous liquid, all components are preferably mixed again, for example by using a high shear mixer, in order to ensure proper homogenization of the functional agents into the matrix components.

Then, the resulting mixture is dried, and, if necessary (depending on the drying technology applied) granulated to obtain the capsules of the present invention.

Drying may be performed by spray drying, freeze drying, fluidized bed drying and/or oven drying, for example. Preferably, the drying step is performed by spray drying.

### EXAMPLES

### Example 1

The retention of different functional agents having different octanol/water partition coefficients (clogp) in capsules of the present invention are compared to capsules based on yeast only, corresponding to the encapsulation technologies disclosed in the prior art. Two different types of yeasts where tested, Yeast 1 (dried DCL) and Yeast 2 (washed “Williams”), commercially obtainable from Lesaffre, France and Aventine Renewable Energy Company, USA, respectively.

Materials

TABLE 2
Sample Recipes

| | | | Maltodextrin | Mass of |
|---|---|---|---|---|
| | Yeast | Flavor | 18DE | water |
| Encapsulation type | (g) | (g) | (g) | (g) |
| Yeast 1 | 150 | 75 | 0 | 375 |
| Yeast 2 | 150 | 75 | 0 | 375 |
| Yeast 1 + Matrix | 150 | 75 | 150 | 375 |
| component* (invention) | | | | |
| Yeast 2 + Matrix | 150 | 75 | 150 | 375 |
| component* (invention) | | | | |

*Maltodextrin DE 18

TABLE 3
Encapsulatable material (flavors)

| Example | Functional agent | ClogP | FIG. |
|---|---|---|---|
| 1 | (+−)-3-HYDROXY-2-BUTANONE | −0.5 | 1 |
| 2 | (+−)-TETRAHYDRO-2-METHYL-3- | 0.83 | 1 |
| | FURANTHIOL | | |
| 3 | 1-(PYRAZINYL)-1-ETHANONE | 0.33 | 1 |
| 4 | S-(2-METHYL-3-FURYL) | 2.11 | 1 |
| | ETHANETHIOATE | | |
| 5 | 4-HYDROXY-2,5-DIMETHYL- | 0.28 | 2 |
| | 3(2H)-FURANONE | | |
| 6 | 1,2,3-PROPANETRIYL | −1.36 | 2 |
| | TRIACETATE | | |
| 7 | Oleic acid | 7.74 | 2 |

All flavoring agents can be commercially obtained in purified form. For each flavor one sample was encapsulated in yeast 1 and 2 alone followed by a washing step and immediate spray drying, and a corresponding sample was made following the process of the present invention, by adding a matrix component (maltodextrin) without any washing step prior to atomization (spray drying).

The yeast was dispersed in water in a 1 liter flask.

The liquid flavor is then added and the mixture is maintained for 4 hours at 50° C. under constant agitation at 150 rpm using a flat blade stirrer.

Process without the use of a matrix component (prior art)

The mixture (water +yeast +flavor) is being separated for 20 minutes in a bench top centrifuge at a speed of 3,200 rpm. The temperature of the centrifuge is maintained at 4° C. The recovered yeast paste was washed twice with distilled water (1,200-1,400ml distilled water) and re-centrifuged (to ensure that all excess active and extraneous material was removed). The yeast cake was then removed from the centrifuge pots and prepared for spray drying.

Distilled water 300 g was added to the yeast cake and mixed until a homogenous dispersion was formed. The samples were then spray dried on a Niro mobile minor at 210° C. inlet and 90-100° C. outlet at a feed rate of approximately 10 ml/minute.

Process according to the invention with addition of a matrix component (Maltodextrin 18DE)

After the 4 hours during which the flavor is being absorbed in the yeast, maltodextrin was added to the encapsulation mixture directly in the flask and mixed until homogenous.

The mixture was then spray dried as such on a Niro mobile minor at 210° C. inlet and 90-100° C. outlet at a feed rate of approximately 10 ml/minute. A powder containing the capsules of the invention is obtained.

Analysis of the Samples

The flavors were isolated from the capsules by extraction with ethanol. In particular, 500 mg of capsules where hydrated with 1 ml water and then mixed with 9 ml ethanol. The suspension was agitated for 10 min, centrifuged and filtered. The filtered liquid was analysed by GS-MS (gas chromatography mass spectrometry), SIM method (Selected Ion Monitoring) in the Split mode.

Results and Conclusions

The results are illustrated in FIGS. 1-2 below, which show the percentage of the different flavors recovered from the different capsules (wt.-% of the flavor used in the preparation). The term MC means matrix component.

In FIG. 1, the recovery of (+−)-3-HYDROXY-2-BUTANONE, tetrahydromethylfuranthiol, 1-(PYRAZINYL)-1-ETHANONE, and S-(2-METHYL-3-FURYL) ETHANETHIOATE from the capsules of Yeast 1 alone, yeast 1 with matrix-component (invention), yeast 2 alone, yeast 2 with matrix component (invention), is given. All four molecules have relatively low clogP values (hydrophilic) and are not well absorbed in yeast cell alone. The addition of the matrix component helps increasing their loading in the final capsules. The use of a matrix component thus increases significantly the flavor recovery from the capsules.

In FIG. 2, the recovery of 4-HYDROXY-2,5-DIMETHYL-3(2H)-FURANONE, 1,2,3-PROPANETRIYL TRIACETATE and oleic acid from the capsules is shown.

It can be seen that the flavor compounds are better retained when a matrix component was added before spray drying. Especially, the amount of 4-HYDROXY-2,5-DIMETHYL-3(2H)-FURANONE and 1,2,3-PROPANETRIYL TRIACETATE (hydrophilic, clogP<1) was clearly higher in capsules comprising a matrix component.

In conclusion, with the encapsulation of several flavors covering similar or varying clogP values, the retention of flavors was more complete in the capsules of the present invention, due to the retention of more hydrophilic flavors in the matrix component.

The matrix component could thus be used, in combination with a micro-organism to effectively encapsulate functional molecules having a clogP of <3, in addition to optional more hydrophobic functional agents, which may be present, too.

### Example 2

For assessing the relevance of hydrophobicity/hydrophilicity of flavor compounds for encapsulation in yeast in absence of a matrix component, the encapsulation efficiency of a total of 140 different flavor compounds of current use in the flavor industry was investigated. The 140 flavors were split in 10 groups of similar chemical classes to form 10 different compositions, each compositions containing 7-19 different compounds. The chemical classes thus grouped together were: (1) acids, furanones and lactones, (2) alcohols and phenols, (3) aldehydes, (4) pyrazines, (5) amines, kenolines, kenoxalines pyridine thiazole, dithiazine, bicyclic lactones, and benzopyrones, (6) ketones and methyl-ketones, (7) sulfide, disulfides, trisulfides and isothiocyanates, (8) esters and thioesters, (9) terpenes and terpene esters, (10) thioles and thiophenes. The different compositions contained from 7 to 19 different compounds. For internal control, each composition contained one flavor compound of a different chemical class. This allowed assessing if the chemical class had an effect on encapsulation efficiency.

For each chemical class, one composition containing equal amounts (5 wt. %) of 7-19 different flavor compounds in equal dilution was thus prepared. Each composition further contained triacetin, to make up 100 wt. % of each flavor composition. The composition with 19 different compounds contained 5 wt. % triacetin.

In this way, 10 flavor compositions encompassing all in all 140 different flavor compounds were prepared.

Each of the 10 compositions spanned a large clogP range. Amongst the 140 compounds, the specimen with the lowest clogP value (-1.09) was diacetyl, and the compound with the highest clogP value (+6.39) was caryophylene ((−)-(1R,9S,E)-4,11,11-trimethyl-8-methylene-bicyclo[7.2.0]undec-4-ene)), as calculated by the method of Suzuki (1992).

Yeast was encapsulated by mixing each flavor composition, dried yeast and water in relative amounts of 12:100:220 under conditions as described in Example 1 (Process without use of a matrix component).

All samples were analysed by ethanol extraction following the procedure given in Example 1 (Analysis of samples).

The encapsulation of efficiency for each flavor compound was calculated by dividing the amount of flavor detected by GC-MS divided by the amount of liquid flavor used for encapsulating.

The results are indicated in FIG. 3, which shows the encapsulation efficiency for each flavor compound as a function of the clogP value. The figure clearly shows a sygmoidal curve with an inflection point between clogP 2 and 3. In other words, compounds with a logP value and lower <3 or even <2 will be increasingly difficult to encapsulate with yeast based systems alone. FIGS. 1 and 2, on the other hand, show that these compounds may well be encapsulated if a matrix component is present, as required by the present invention.

What is claimed is:

1. Capsules comprising a micro-organism, a matrix component, and, at least one encapsulatable material, whereby the matrix component and the encapsulatable material do not originate from the micro-organism itself, and whereby the encapsulatable material comprises at least one functional agent that is characterized by a calculated octanol/water partition coefficient clogP less than 3.

2. The capsules according to claim 1, comprising at least one additional, other functional agent, which is characterized by an calculated octanol/water partition coefficient clogP of 1 or higher.

3. The capsules according to claim 1, wherein the functional agent is characterized by a clogP of smaller than 2.

4. The capsules according to claim 2, wherein the additional functional agent has a clogP of 2 or higher.

5. The capsules according to claim 1, wherein the encapsulatable material further comprises a carrier.

6. The capsules according to claim 1, wherein the micro-organism provides 5 to 80%, the matrix component provides 5 to 80% and the encapsulatable material comprising at least one functional agent provides 5 to 60% of the dry weight of the capsule.

7. The capsules according to claim 1, wherein the matrix component comprises a water soluble carbohydrate.

8. A delivery system comprising the capsules according to claim 1.

9. A food product comprising the capsules of claim 1.

10. A process for preparing the capsules of claim 1, comprising the steps of
preparing an aqueous liquid comprising at least a micro-organism and water,
adding a encapsulatable material comprising a functional agent having a clogP of smaller than 3,
optionally adding a further encapsulatable material comprising a functional agent having a clogP of 1 or higher
stirring, agitating or mixing the aqueous liquid and the encapsulatable material(s),
adding a matrix component
drying the components, and
optionally granulating the dried slurry to obtain the capsules.

11. A process for preparing a delivery system which comprises preparing the capsules according to claim 10.

12. A process for preparing a food product which comprises preparing the capsules according to claim 10.
